(19) United States
(12) Patent Application Publication
(10) Pub. No.: US 2017/0079591 A1
(43) Pub. Date: Mar. 23, 2017

(54) SYSTEM AND METHOD FOR OBTAINING VITAL MEASUREMENTS USING A MOBILE DEVICE

(71) Applicant: QUALCOMM Incorporated, San Diego, CA (US)

(72) Inventors: Russell GRUHLKE, Milpitas, CA (US); Igor TCHERTKOV, San Jose, CA (US); Russel Allyn MARTIN, Menlo Park, CA (US); Evgeni POLIAKOV, San Mateo, CA (US); Evgeni GOUSEV, Saratoga, CA (US); Liang SHEN, Toronto (CA); Alok GOVIL, Fremont, CA (US)

(21) Appl. No.: 14/860,645

(22) Filed: Sep. 21, 2015

(51) Int. Cl. A61B 5/00; A61B 5/0205; A61B 5/0408; A61B 5/021; A61B 5/026; A61B 5/0295

(52) U.S. Cl. CPC A61B 5/6898; A61B 5/0261; A61B 5/0295; A61B 5/0408; A61B 5/021; A61B 5/7278; A61B 5/742; A61B 5/681; A61B 5/0205

(57) ABSTRACT

Methods, systems, computer-readable media, and apparatuses for obtaining vital measurements are presented. The vital measurements may include a blood pressure value that can be obtained by determining a pulse-transit time (PTT) as a function of a photoplethysmography (PPG) measurement and electrocardiogram (ECG) measurement. A mobile device includes an outer body sized to be portable for a user, a processor contained within the outer body, a display coupled to a light guide, and at least one first sensor coupled to the light guide. The display is configured to display an illumination pattern directing light toward blood vessels within the user. The at least one first sensor is configured to measure reflected light from the illumination pattern reflected off of the blood vessels within the user, wherein the processor is configured to obtain a first measurement indicative of changes in blood volume based at least in part on the measured reflected light.

### BACKGROUND

Aspects of the disclosure relate to mobile devices, and more particularly, a system and method for obtaining vital measurements of a user operating a mobile device.

It is often desirable for a user to be aware his/her vital measurements (e.g., bodily function measurements). Recently, many individuals wear small portable devices capable of measuring their heart rate (HR) and blood pressure (BP). Both HR and BP are measurements that can reveal vital information about an individual's health, fitness, and emotional state. Obtaining an HR measurement is relatively simple and involves counting the number of pulses palpated in a unit of time. Established methods of measuring HR include electrocardiogram (ECG) and photoplethysmogram (PPG). In contrast, obtaining a BP measurement typically requires an inflatable cuff. Additionally, many small devices can estimate BP by using the pulse transit time (PTT) technique, which computes the delay time between an ECG heartbeat and a PPG blood pulse. However, these small devices still have many shortcomings.

PTT based BP estimations that are implemented on mobile devices (e.g., smartphones and smart watches) add additional complexity and material cost for device manufacturers. Consumers are typically not willing to pay extra for these features, and expect them to already be a part of the default feature set of the device. Further, they require additional real estate within the device itself, increasing design complexity for the device manufacturers often resulting in less than desirable device form-factors.

Accordingly, a need exists for a small mobile device that can provide HR and BP measurements that leverages hardware that is inherit on such devices.

### BRIEF SUMMARY

Certain implementations are described for obtaining at least one bodily function measurement of a user operating a mobile device.

In some implementations, a mobile device for obtaining vital measurements includes an outer body sized to be portable for a user of the mobile device, a processor contained within the outer body, a display coupled to a light guide, and at least one first sensor coupled to the light guide. The display may be configured to display an illumination pattern directing light toward blood vessels within the user. The at least one first sensor may be configured to measure reflected light from the illumination pattern reflected off of the blood vessels within the user. The processor may be configured to obtain a first measurement indicative of changes in blood volume based at least in part on the measured reflected light.

In some implementations, the mobile device includes at least one second sensor coupled to the outer body, the at least one second sensor configured to obtain a second measurement indicative of heart electrical activity.

In some implementations, the second measurement indicative of heart electrical activity comprises an electrocardiography (ECG) measurement.

In some implementations, the processor is further configured to facilitate generation of a blood pressure value based on the first measurement and the second measurement.

In some implementations, the at least one first sensor comprises a photodiode.

In some implementations, the at least one second sensor comprises at least a first electrode and a second electrode, and wherein a portion of the user's body completes a circuit between the first electrode and the second electrode.

In some implementations, the first measurement indicative of changes in blood volume comprises a photoplethysmography (PPG) measurement.

In some implementations, the mobile device is at least one of a smartphone device or a watch.

In some implementations, a method for obtaining vital measurements includes displaying, via a display device, an illumination pattern directing light toward blood vessels within a user of a mobile device, wherein the display device is coupled to a light guide, and wherein the mobile device comprises an outer body sized to be portable for the user. The method also includes measuring, via a first sensor coupled to the light guide, reflected light from the illumination pattern reflected off of the blood vessels within the user. The method additionally includes obtaining, via a processor, a first measurement indicative of changes in blood volume based at least in part on the measured reflected light.

In some implementations, an apparatus for obtaining vital measurements includes means for displaying, via a display device, an illumination pattern directing light toward blood vessels within a user of a mobile device, wherein the display device is coupled to a light guide, and wherein the mobile device comprises an outer body sized to be portable for the user. The apparatus also includes measuring, via a first sensor coupled to the light guide, reflected light from the illumination pattern reflected off of the blood vessels within the user. The apparatus additionally includes obtaining, via a processor, a first measurement indicative of changes in blood volume based at least in part on the measured reflected light.

In some implementations, one or more non-transitory computer-readable media store computer-executable instructions for obtaining vital measurements that, when executed, cause one or more computing devices included in a mobile device to display, via a display device, an illumination pattern directing light toward blood vessels within a user of a mobile device, wherein the display device is coupled to a light guide, and wherein the mobile device comprises an outer body sized to be portable for the user. The instructions, when executed, also cause the one or more computing devices to measure, via a first sensor coupled to the light guide, reflected light from the illumination pattern reflected off of the blood vessels within the user. The instructions, when executed, also cause the one or more computing devices to obtain, via a processor, a first measurement indicative of changes in blood volume based at least in part on the measured reflected light.

### BRIEF DESCRIPTION OF THE DRAWINGS

Aspects of the disclosure are illustrated by way of example. In the accompanying figures, like reference numbers indicate similar elements, and:

FIG. 1 illustrates a simplified block diagram of a mobile device that may incorporate one or more implementations;

FIG. 2 illustrates a smartphone device configured to obtain PPG and ECG measurements of a user, according to some embodiments;

FIG. 3 illustrates a smartphone device having two contacts and an illuminated image shown on the display, according to some implementations;

FIG. 4 illustrates cross-sectional view of display and a cover glass, according to some implementations;

FIG. 5A illustrates a top-view of a display not having a cover glass with light guiding features, according to some implementations;

FIG. 5B illustrates a top-view of a display having a cover glass with light guiding features, according to some implementations;

FIG. 6 illustrates a top view of a touchscreen display having a plurality of light sensors coupled to edges of the cover glass, according to some implementations;

FIG. 7 illustrates a timing diagram for a pulsed touchscreen display light source, according to some implementations;

FIG. 8 is a flowchart of a method for obtaining vital measurements, according to some implementations; and

FIG. 9 illustrates an example of a computing system in which one or more embodiments may be implemented.

### DETAILED DESCRIPTION

Several illustrative implementations will now be described with respect to the accompanying drawings, which form a part hereof. While particular implementations, in which one or more aspects of the disclosure may be implemented, are described below, other implementations may be used and various modifications may be made without departing from the scope of the disclosure or the spirit of the appended claims.

Some implementations pertain to a small mobile device (e.g., smartphone and smart watch) that typically already includes a liquid crystal display (LCD), or other type of display, for example an emissive display such as an OLED. The LCD display can be leveraged and used as a light source for a PPG-based HR measurement. Alternatively, a reflective display could utilize a backlight to provide additional light if ambient light is insufficient as a light source, once reflected off the display. Additionally, the cover glass of the LCD display can be used as a light guide to direct light toward one or more light sensors (e.g., photodiodes). The mobile device can also include one or more electrodes for completing a circuit through a user's body, in order to obtain an ECG measurement.

The LCD can be configured to display a particular image to assist in obtaining a PPG-based HR measurement. For example, the display can provide an image of two red or green spots that the user can place his/her fingers on (e.g., one finger or thumb from each hand). In other words, the LCD displays an illuminated pattern of a particular color and shape, which is used as a light source for obtaining a PPG measurement. When the user places his/her finger on top of the illuminated pattern being shown on the display, the light may be reflected back into the cover glass of the LCD display. The cover glass may act as a light guide and direct the reflected light toward a light sensor (e.g., photodiode) located a small distance (e.g., 20-35 mm) away from the light source. For example, one or more light sensors may located at the edge of the display while the illuminated pattern may be displayed toward the center of the display. The cover glass of the display may direct the light reflected from the user's finger at the center of the display toward the one or more light sensors located at the edge of the display. In another embodiment, the illuminated pattern may be displayed toward the edges of the display or toward the corners, near light sensors.

The principle described above works because the reflected light from the user's finger may be modulated by arterial pulsations and then may be coupled back into the cover glass. In some cases, since the cover glass is surrounded by air with refractive index of 1 (in contrast with n=1.5 for glass), the light propagating in the cover glass undergoes multiple total internal reflections. In effect, the cover glass surrounded by the air acts as a light-guide allowing HR-modulated reflected light to reach the edge of the cover glass (e.g., edge of the display) where the light sensor (e.g., photo detector) is attached. In other cases, an air gap may not be desirable in display devices since contrast is reduced by Fresnel reflections at a glass/air interface. In such cases, a low index transparent material may be coupled to the light guide such that the light guide is surrounded by air and the low index material.

The user's HR can then be determined from the PPG measurement based on the HR-modulated light detected at the light sensors. Additionally, the ECG sensors located on the small mobile device can be used to measure the user's heartbeat. The user's BP can then be estimated using the PPG measurement and the ECG measurement, by way of the PTT technique.

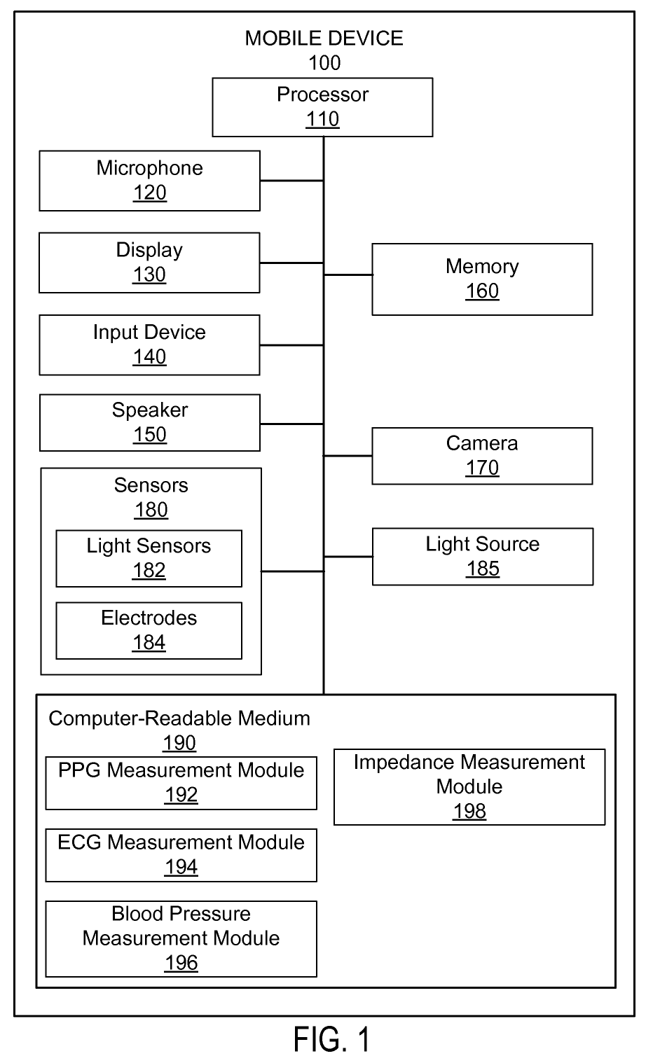

FIG. 1 illustrates a simplified block diagram of a mobile device 100 that may incorporate one or more implementations. Mobile device 100 may include a processor 110, microphone 120, display 130, input device 140, speaker 150, memory 160, camera 170, sensors 180, light source 185, and computer-readable medium 190.

Processor 110 may be any general-purpose processor operable to carry out instructions on the mobile device 100. The processor 110 is coupled to other units of the mobile device 100 including microphone 120, display 130, input device 140, speaker 150, memory 160, camera 170, sensors 180, light source 185, and computer-readable medium 190.

Microphone 120 may be any an acoustic-to-electric transducer or sensor that converts sound into an electrical signal. The microphone 120 may provide functionality for a user of the mobile device 100 to record audio or issue voice commands for the mobile device 100.

Display 130 may be any device that displays information to a user. Examples may include an LCD screen, CRT monitor, or seven-segment display.

Input device 140 may be any device that accepts input from a user. Examples may include a keyboard, keypad, or a mouse. In some implementations, the microphone 120 may also function as an input device 140.

Speaker 150 may be any device that outputs sound to a user. Examples may include a built-in speaker or any other device that produces sound in response to an electrical audio signal and/or ultrasonic signal(s).

Memory 160 may be any magnetic, electronic, or optical memory. It can be appreciated that memory 160 may include any number of memory modules. An example of memory 160 may be dynamic random access memory (DRAM).

Camera 170 is configured to capture one or more images via a lens located on the body of mobile device 100. The captured images may be still images or video images. The camera 170 may include a CMOS image sensor to capture the images. Various applications running on processor 110 may have access to camera 170 to capture images. It can be appreciated that camera 170 can continuously capture images without the images actually being stored within the mobile device 100. Captured images may also be referred to as image frames.

Sensors 180 may be a plurality of sensors configured to obtain data accessible by the processor. The sensors 180 may also be physically coupled to the outer body of the mobile device 100. The plurality of sensors 180 may include one or more light sensors 182 and/or one or more electrodes 184. The light sensors 182 may be configured to facilitate measurement of reflected light from the light source 185 (described below) reflected off of blood vessels within a user of the mobile device 100 to obtain the a PPG measurement indicative of changes in the user's blood volume. Light sensors 182 may be referred to as light collecting components. The light sensors 182 may include one or more photodiodes. A portion of a user of the mobile device's 100 body may complete a circuit between a first electrode and a second electrode, e.g., when the user touches both electrodes 184. The electrodes 184 may be configured to facilitate measurement of heart electrical activity of the user to obtain an ECG measurement.

Light source 185 may be any source of light configured to emit light through a user's body. In some implementations, the light source 185 may be a emitted via the display 130 of the mobile device 100. The emitted light may be of a wavelength that can pass through parts of a user's body. For example, the light source 185 may be an LED emitting light through a user's wrist. The light emitted from light source 185 may reflect off of blood vessels within the user's body and the reflected light may be measured by one or more light sensors 182 to obtain a PPG measurement, as described above. It can be appreciated that emitted light may be of different wavelengths depending on different variables. For example, different wavelengths of light may be appropriate to improve the signal, reduce noise, deal with dark skin colors, measure the blood's oxygen content, or penetrate to different depths of the user's body. Light source 185 may also be referred to as a light emitting component.

Computer-readable medium 190 may be any magnetic, electronic, optical, or other computer-readable storage medium. Computer-readable medium 190 includes PPG measurement module 192, ECG measurement module 194, blood pressure value module 196, and impedance measurement module 198.

PPG measurement module 192 is configured to, when executed by processor 110, obtain a photoplethysmography (PPG) measurement. The PPG measurement may be a measurement of changes in blood volume of a user operating the mobile device 100. The PPG measurement may be obtained by the PPG measurement module 192 in response to a user action. The PPG measurement module 192 may interface with the light source 185 and light sensors 182 in order to obtain the PPG measurement. Upon indication by the user of a need for a PPG measurement, the PPG measurement module 192 may direct the light source 185, or multiple light sources, to emit light through the user's body. As described above, the emitted light may reflect off or be transmitted through blood vessels within the user's body and may be detected by one or more light sensors 182 within the mobile device 100. The PPG measurement module 192 may measure, by interfacing with the one or more light sensors, the amount of reflected or transmitted light detected by the one or more light sensors 182. The PPG measurement module 192 may then determine a PPG measurement that is indicative of changes in the user's blood volume based on the measurement of the reflected light.

ECG measurement module 194 is configured to, when executed by processor 110, obtain an electrocardiography (ECG) measurement. The ECG measurement may be a measurement of heart electrical activity of a user operating the mobile device 100. The ECG measurement may be obtained by the ECG measurement module 194 in response to a user action. The ECG measurement module 194 may interface with the electrodes 184 in order to obtain the ECG measurement. Upon indication by the user of a need for an ECG measurement, the ECG measurement module 194 may interface with the electrodes 184 to measure (assuming the user's body completes a circuit between the electrodes 184) electrical impulse(s) generated by the polarization and depolarization of cardiac tissue within the user's body. In some implementations, the electrical impulse(s) may be generated by the beating of the user's heart. In some implementations, the ECG measurement module 194 may interface with the electrodes 184 to measure the electrical impulse(s) automatically upon the user's body completing a circuit between the electrodes 184. The ECG measurement module 194 may then determine an ECG measurement based on the measured electrical impulse(s). It can be appreciated that ECG measurement can be obtained using two or more electrode leads.

Blood pressure value module 196 is configured to, when executed by processor 110, generate a blood pressure value of the user based on the PPG measurement and the ECG measurement. According to Poon, C. C. Y.; Zhang, Y. T. “Cuff-less and Noninvasive Measurements of Arterial Blood Pressure by Pulse Transit Time”, Engineering in Medicine and Biology 27^{th} Annual Conference, 2005. IEEE, On page(s): 1-4, the calculation of the blood pressure value based on the PPG measurement and the ECG measurement is well known in the art.

Impedance measurement module 198 is configured to, when executed by processor 110, obtain an impedance measurement. The impedance measurement may be indicative of a hydration level of a user operating the mobile device 100. The impedance measurement may be obtained by the impedance measurement module 198 in response to a user action. The impedance measurement module 198 may interface with the electrodes 184 in order to obtain the impedance measurement. Upon indication by the user of a need for an impedance measurement, the impedance measurement module 198 may interface with the electrodes 184 to measure (assuming the user's body completes a circuit between the electrodes 184) electrical impedance through the user's body. In some embodiments, the impedance measurement module 198 may interface with the electrodes 184 to measure the electrical impedance automatically upon the user's body completing a circuit between the electrodes 184.

It can be appreciated that the mobile device 100 may be sized to be portable for a user. It can be appreciated that the term “portable” may refer to something that is able to be easily carried or moved, and may be a light and/or small. The term portable may refer to something easily transportable by the user or wearable by the user. For example, the mobile device 100 may be a smartphone device or a watch wearable by the user. Other examples of portable devices include a head-mounted display, calculator, portable media player, digital camera, pager, personal navigation device, electronic reader (e-reader) etc. Examples of devices that may not be considered portable include a desktop computer, traditional telephone, television (not including a portable television or display system for watching movies, such as a DVD player), appliances, etc. It can be appreciated that the bodily function measurements can be obtained via the smartphone, watch, or any other of the mentioned devices.

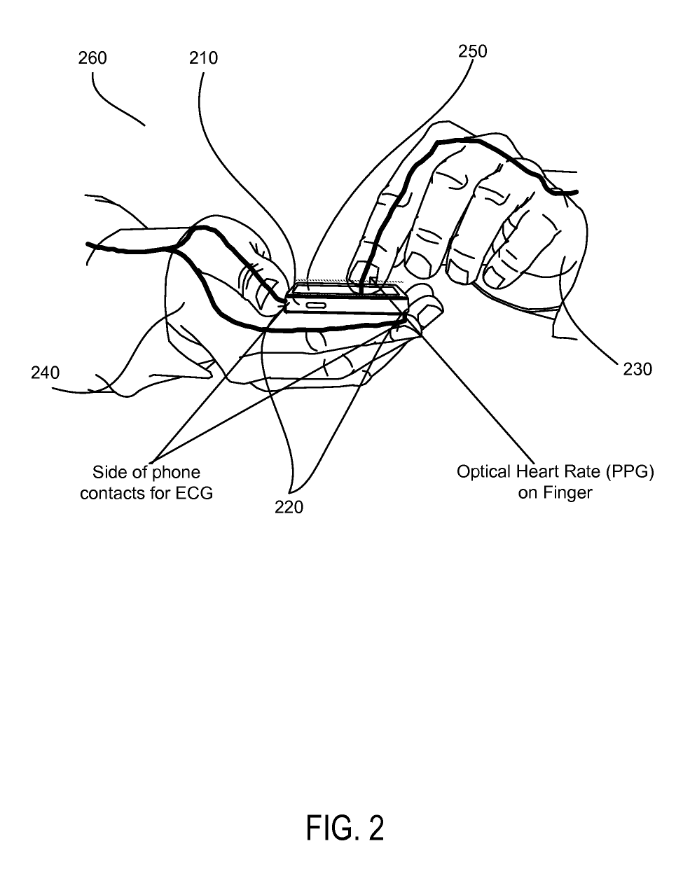

FIG. 2 illustrates a smartphone device 210 configured to obtain PPG and ECG measurements of a user, according to some embodiments. It can be appreciated that the smartphone device 210 is only one example of a mobile device 100 and other equally suitable types of portable devices include an e-reader, personal digital assistant (PDA), DVD player, etc. The smartphone device 210 may include a plurality of contacts 220. In some embodiments, a single contact 220 may be positioned at each end of the smartphone device 210. In other embodiments, a touchscreen display 250 of the smartphone device 210 may include a contact layer including, e.g., silver metal or Indium Tin Oxide (ITO). The smartphone device 210 may obtain both PPG and ECG measurements of the user 260.

For example, the user 260 may hold the smartphone device 210 with his/her first hand 240 touching one or more of the contacts 220 and with his/her second hand 230 touching the touchscreen display 250. Upon the user 260 performing this action, the contacts 220 and the contact layer of the touchscreen display 250 may complete a circuit through the user's 260 body. The smartphone device 210 may then measure an electrical potential through the completed circuit to determine the ECG measurement. It can be appreciated that the ECG measurement may also be obtained without the user's first hand 240 or second hand 230 contacting the touchscreen display 250. That is, the user's first hand 240 may make contact with a first side contact 220 and the user's second hand 230 may make contact with a second side contact 220 to complete the circuit. Alternatively, the user 260 may make contact with both side contacts 220 using only his/her first hand 240 or second hand 230 (see below for a measurement of PPG or Galvanic Skin Response (GSR)). Alternatively, and not illustrated in FIG. 1, sensors positioned and/or touched at other locations, for example legs, feet, ankles, knees, elbows, arms, neck, head, etc. could also be used to generate PPG, GSR and possibly ECG, depending on the location and how the contact was made. For example, a watch being worn on the user's wrist or a head-mounted device such as glasses (not illustrated) could include the sensors needed to generate some or all the information needed.

The touchscreen display 250 of the smartphone device 210 may also obtain a PPG measurement of the user 260 by using an optical based technology. For example, when the user 260 touches the touchscreen display 250, the touchscreen display may generate a light that shines into the user's 260 skin, measure the blood flow through the capillaries and thus determine a heart rate (PPG) of the user. It can be appreciated that the touchscreen display may generate the light using elements built-in to the display without the need for discrete optical light sources. This process is described in further detail below.

Accordingly, by obtaining both the PPG and ECG measurements of the user 260, a PTT technique may be used to determine the user's blood pressure. The smartphone device 210 may then provide important information to the user 260, based on the determined blood pressure (described further below).

Additionally, the smartphone device 210 may obtain an impedance measurement of the user using Bioelectrical Impedance Analysis (BIA) techniques. In some embodiments, the impedance measurement may be obtained via the contact layer of the touchscreen display 250. The process of obtaining the impedance measurement is described in further detail below.

It can be appreciated that the touchscreen display 250 may serve multiple functions. That is, the touchscreen display 250 may be used to obtain ECG, PPG, and/or impedance measurements as described above, and may also be used as a user input device. The user 260 may use the touchscreen display 250 to provide input to applications being executed on the smartphone device 210. When the user 260 wishes to obtain a bodily function measurement using the touchscreen display 250, the user 260 may place the smartphone device 210 into a measurement mode. Alternatively, the smartphone device 210 may automatically detect the user's intention to obtain a bodily function measurement, e.g., from the user 260 placing his/her finger in a particular location on the touchscreen display 250 or touching the touchscreen display 250 for a predetermined period of time. Alternatively, the smartphone device 210 may regularly scan and store vital signs of the user 260 in the user's normal course of operating the smartphone device 210, without the user wanting or needed a particular vital sign report at that time, and without the user prompting each measurement.

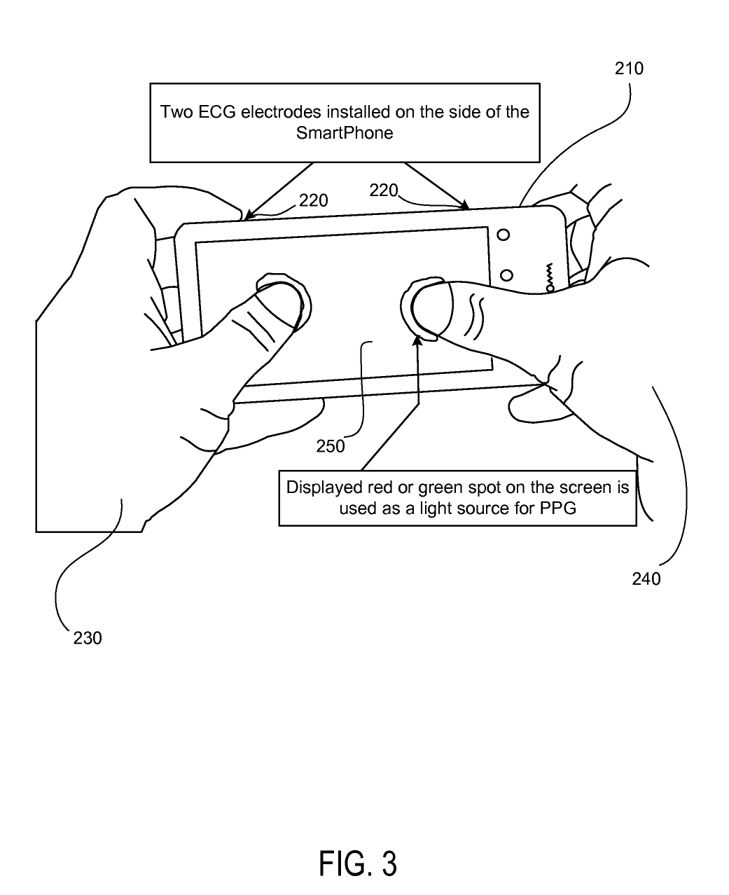

FIG. 3 illustrates a smartphone device having two contacts and an illuminated image shown on the display, according to some implementations. The figure shows two side contacts 220 (e.g., electrodes) that may make contact with the user's fingers in order for the smartphone device 210 to obtain an ECG measurement of the user. The side contacts 220 may be located on either the side of the back of the smartphone device 210. In some implementations, the contacts 220 may be placed in other locations on the smartphone device 210, e.g., at the bottom of the smartphone device 210. The contacts 220 can be positioned anywhere such that the user is able to complete a circuit through his/her body using the contacts 220.

Additionally, the touchscreen display 250 may generate a light source that can be used in order to shine light into the user's arteries in order to measure to the reflected light to obtain a PPG measurement. In some implementations, the generated light may be red or green in color and may be of a particular wavelength. The generated light may be generated by elements of the touchscreen display 250 itself without the need for a discrete light source. For example, a group of pixels within the touchscreen display 250 may be controlled to display red light in the locations where the user's thumbs are shown. It can be appreciated that since the generated light is constant, e.g. not changing colors, the modulation of the LCD display is trivial and basically the refresh rate of the touchscreen display 250. In another implementation, a display lacking touchscreen functionality may be used.

Additionally, one or more light sensors may be glued to the edge of the cover glass of the touchscreen display 250. In some implementations, the light sensors may be photodiodes. The light sensors may function to detect light that is reflected off the user's finger(s) generated by the touchscreen display 250. The reflected light may be directed toward the light sensors via a light guide that is attached to the cover glass of the touchscreen display 250. For example, four photodiodes may be glued to the side surface of the cover glass of the touchscreen display 250, with one photodiode on each side. The photodiodes may also be mounted in other manners, for example built-in, soldered, etc. The function of the light guide is described in further detail below.

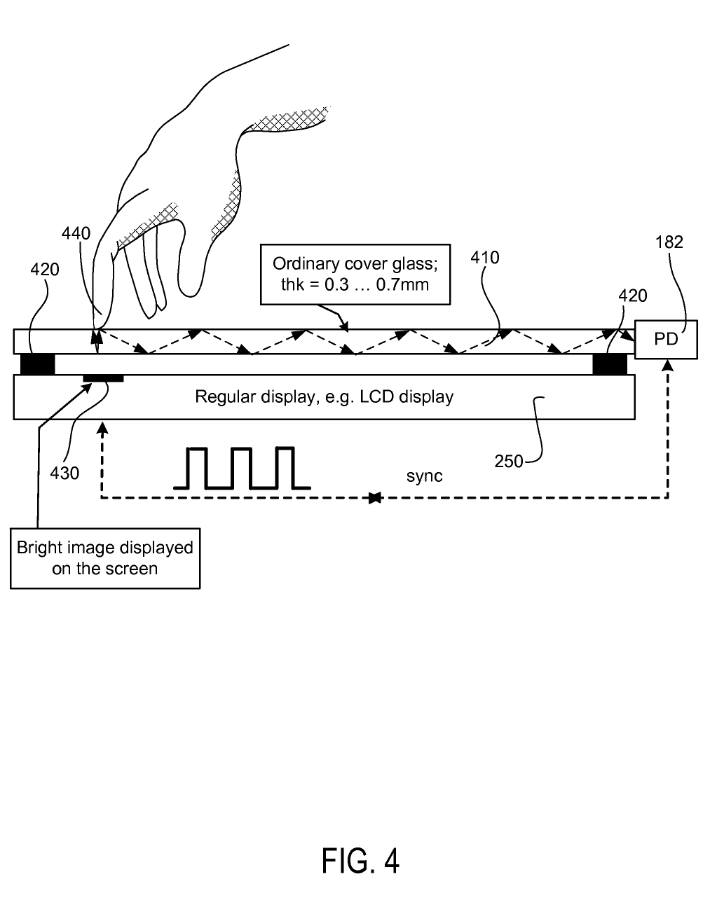

FIG. 4 illustrates a cross-sectional view of a display and a cover glass, according to some implementations. As described above, a cover glass 410 is attached to the touchscreen display 250. The cover glass 410 may be attached to the touchscreen display 250 via one or more spacers 420 configured to separate the cover glass 410 from the touchscreen display 250 by a nominal distance, e.g., just a few millimeters. The cover glass 410 may be glued to the spacers 420, which in turn may be glued to the touchscreen display 250. In some implementations, a low index adhesive (not shown) may be used instead of the spacers 420. In some embodiments, a coupling material (not shown) may fill some or all the gap between the touchscreen display 250 and cover glass 410 with an index of refraction appropriate to the materials in either layer to enable total internal reflection within the cover glass 410.

The touchscreen display 250, in response to an instruction from the processor, may generate light in the form of a colored image displayed by a particular group of pixels within the touchscreen display 250. The particular group of pixels may be located in a position where the user is expected to touch his/her finger to the touchscreen display 250. In some implementations, the colored image 430 may be red or green in color. Once the user touches his/her finger 440 to the touchscreen display 250 (e.g., via the cover glass 410), the light from the colored image 430 directed toward the user's finger 440 is modulated by the arterial pulsation within the finger and the light is coupled back into the cover glass 410. In some implementations, the cover glass may be between 0.3 mm and 0.7 mm in thickness. Once the light directed toward the user's finger 440 is reflected back into the cover glass 410, the light propagates within the cover glass 410 while undergoing multiple total internal reflections. This may occur because the cover glass 410 may be surrounded by air with a refractive index of 1 (in contrast with n=1.4 for the cover glass 410). In effect, the cover glass 410 may act as a “light-guide” allowing the heart rate-modulated reflected light to reach the edge of the cover glass 410 where a light sensor 182 (e.g., photodiode) is located. The spacers 420 may allow for an “air gap” in between the cover glass 410 and the touchscreen display 250 such that a different refractive index exists. It can be appreciated that even though the light source (e.g., colored pixels on the touchscreen display 250) and the light sensor 182 are some distance apart, the light sensor 182 may still measure the light reflected off of the user's finger 440 due to the total internal reflections suffered by the light. This may be in contrast to existing solutions where the light sensor must be positioned only a few millimeters from the light source in order to obtain an accurate measurement of reflected light. Implementations described herein may allow for the light sensor to be positioned at a significant distance (e.g., 30 mm) away from the light source and still obtain an accurate measurement of reflected light.

Upon the light sensor 182 measuring the amount of light detected, the measurement may be used by the processor of the smartphone device 210 to determine a PPG measurement for the user. Additionally, the light sensor 182 and the color image displayed via the pixels on the touchscreen display 250 may be synchronized to mitigate any ambient light pollution affecting the measurements of the light sensor 182. In essence, the light sensor 182 may measure the light twice. The light sensor 182 may measure the light when the light source (e.g., the colored image displayed via the pixels on the touchscreen display 250) is active, and once when the light source is not active. Effectively, the colored image showing the touchscreen display 250 is pulsed at a particular frequency equal to the synchronization frequency (e.g., the refresh rate of the display 250). Therefore, when the light source is not active, the light sensor 182 may be measuring the ambient light pollution. When the light source is active, the light sensor 182 may be measuring both the reflected light from the user's finger (e.g., the useful signal) and the ambient light pollution together. The measurement when the light source is not active may be removed from the measurement when the light source is active to obtain the measured light from the user's finger (e.g., the useful signal) only. The synchronization may be performed at a frequency equal to the refresh rate of the touchscreen display 250, or may be performed at a frequency that is entirely different. It can be appreciated that the synchronization need not be tied to the refresh rate for purposes of any measurement, but may be tied to the refresh rate for if so constrained by design requirements of the display.

In some implementations, the user may touch the touchscreen display 250 with two fingers on the same hand. The touchscreen display 250 may display colored images in two separate areas of the display. The user may touch a first finger in the first area and a second finger in the second area simultaneously. Due to a slight difference in a blood flow path between the two fingers, the PPG signals (e.g., measured reflected light) generated at the two different areas may have a small phase difference that can be measured. From this measurement, information about the vasculature condition of the user may be extracted. For example, the tip of the user's middle finger may be located approximately 50 mm to 70 mm farther away from the heart than the user's thumb on the same hand. The blood flow path length difference may allow for PTT measurements from which BP can be extracted.

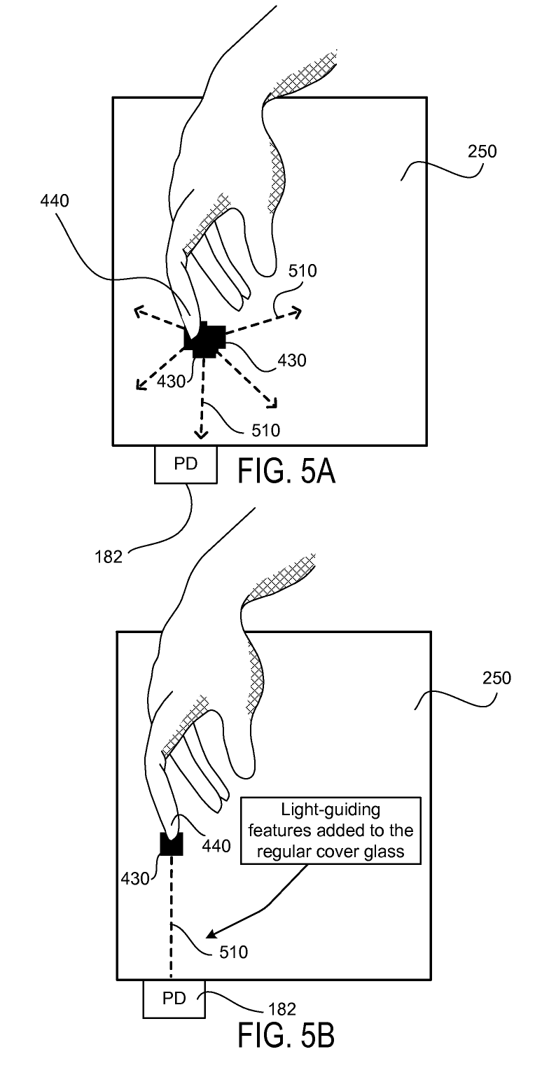

FIG. 5A illustrates a top-view of a display not having a cover glass with light guiding features, according to some implementations. The figure shows a top view of a touchscreen display 250 with a user's finger 440 contacting the touchscreen display 250 at a location where a colored image 430 is generated. However, the cover glass (not shown) above the touchscreen display 250 does not include any light guiding features. As can be seen, the reflected light 510 reflected from the user's finger 440 is scattered in a variety of directions across the touchscreen display 250. The disadvantage in not having a light guide may be that the scattered reflected light 510 may result in insufficient reflected light 510 detected by the light sensor 182. In turn, the light sensor 182 may not be able to provide an accurate measurement of the reflected light 510 reflected off of the user's finger 440. Therefore, an accurate PPG measurement for the user may not be able to be determined.

In contrast, FIG. 5B illustrates a top-view of a display having a cover glass with light guiding features, according to some implementations. The figure shows a top view of a touchscreen display 250 with a user's finger 440 contacting the touchscreen display 250 at a location where the colored image 430 is generated. However, unlike the illustration in FIG. 5A, the cover glass (not shown) may include light guiding features. As a result, the reflected light 510 reflected from the user's finger 440 may be guided in a direction toward the light sensor 182. The advantage to having light guiding features in the cover glass may be so that a larger portion of the useful signal (e.g., the reflected light 510) is detected by the light sensor 182. In turn, the light sensor may be able to provide an accurate measurement of the reflected light 510 reflected off the user's finger 440 and the processor of the smartphone device may be able to determine an accurate PPG measurement for the user.

In some embodiments, the cover glass having light guide features can comprise glass, acrylic (pmma), polycarbonate, PET, etc.

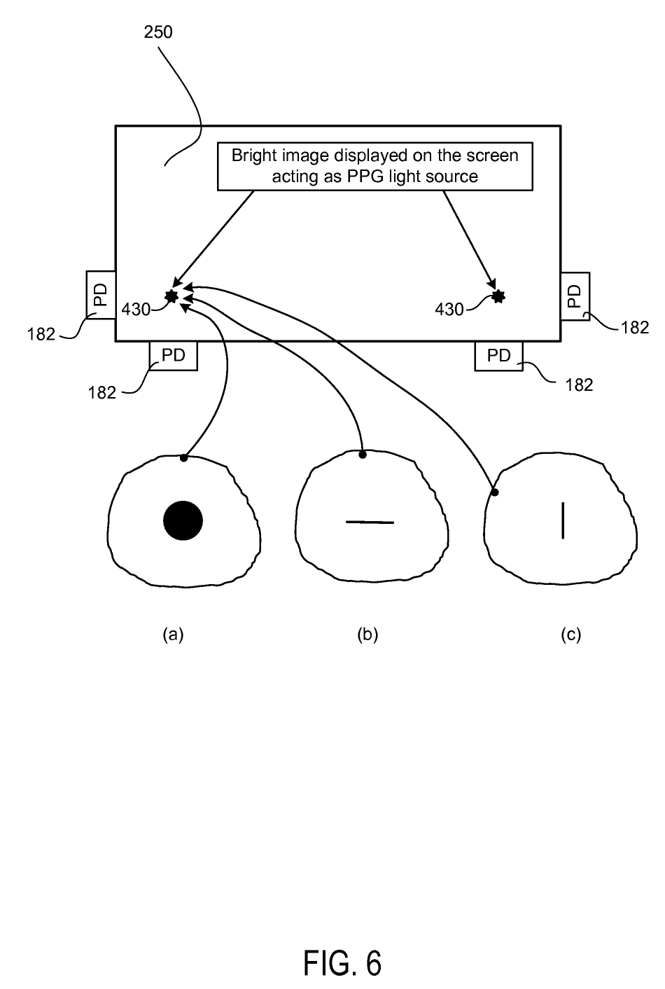

FIG. 6 illustrates a top view of a touchscreen display 250 having a plurality of light sensors 182 coupled to edges of the cover glass, according to some implementations. In some implementations, the light sensors 182 (e.g., photodiodes) may be positioned near two of the four corners of the touchscreen display 250. These locations may be relatively close to the colored images 430 generated via pixel values on the touchscreen display 250. The colored images 430 may be generated at optimal locations for a user to place his/her finger while holding the smartphone device 210 in a particular orientation. For example, the user may hold the smartphone device 210 in a “landscape” orientation, similar to what is shown in FIG. 3. In this orientation, the user may have his/her index finger touching the contacts 220 used for determining the ECG measurement while also having both thumbs touching the touchscreen display 250 (e.g., via the cover glass). The user's thumbs may be positioned over the colored images 430 on the touchscreen display 250.

The colored images 430 may be rendered in a variety of different pixel orientations. In one example, (a) shows the colored image 430 as a round shape rendered by a cluster of pixels. In another example, (b) shows the colored image 430 as a horizontal line rendered by a single row of pixels. In yet another example (c) shows the colored image 430 as a vertical line rendered by a single column of pixels. A variety of other ways of rendering the colored images 430 with the pixels of the touchscreen display 250 may also exist. Different variations of rendering the colored image 430 may provide certain advantages. For example, one variation of rendering the colored images 430 may be rendered within a shorter period of time than another variation of rendering the colored images 430.

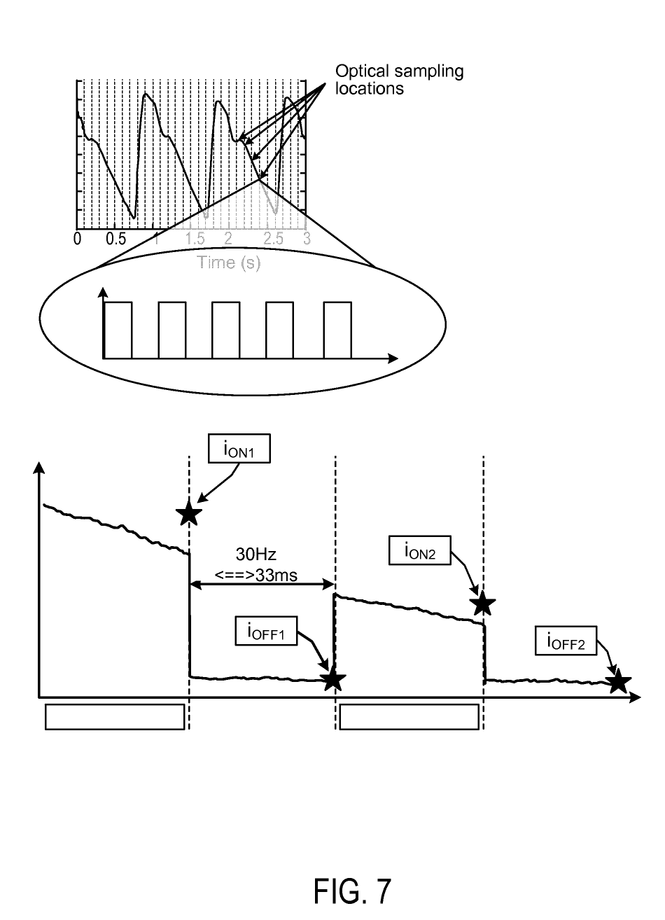

FIG. 7 illustrates a timing diagram for a pulsed touchscreen display light source, according to some implementations. Existing solutions often use a continuous wave (CW) regime for a PPG light source, where the light source is continuously on. The photocurrent (e.g., the reflected light) generated by the reflected light may be modulated intentionally by the HR only. In this mode, the ambient light may add additional illumination, working in “parallel” with the PPG light source. If the ambient light is not constant (which is often the case), e.g., 120 Hz modulation of a fluorescent overhead light, then the photocurrent may also become modulated (in addition to the HR modulation) by the variation in intensity of the ambient light.

In order to mitigate this noise caused by the ambient light, the light source may be pulsed on a pre-determined frequency. As described above, the photocurrent may be measured twice: (a) once during the time with the light source is ON and (b) once during the time when the light source is OFF. The photocurrent measured by the light sensors during the OFF state may be subtracted from the photocurrent measured by the light sensors during the ON state, effectively removing artifacts caused by the variation of ambient illumination from the signal.

In some implementations, the light sensors 182 may be synchronized with the touchscreen display 250 for enhanced performance. Typical displays are automatically refreshed at 30, 60, or 120 Hz. Refreshing the display may cause a very small amount of modulation (e.g., 0.3%) of intensity of a constant image being displayed. This amount may be significantly lower than the typical modulation of reflected light reflected from a user's finger, which is typically around 3% when picked off directly from the finger tip.

Thus, although possible to use the CW regime to constantly display the light source, it may be advantageous to pulse the light source (e.g., the colored images on the touchscreen display 250). The theoretical possible fastest human HR can be estimated as 4 Hz=240 bpm. In order to resolve a signal having such a frequency, according to the Nyquist theorem, the signal must be sampled at a frequency faster than 8 Hz. The slowest refresh rate of a typical touchscreen display 250 may be conservatively set at 30 Hz, which is already several times faster than the minimal Nyquist sampling frequency. Modern touchscreen displays 250 often have higher refresh rates, e.g., up to 120 Hz or higher. Thus, an advantage may exist when pulsing the light source ON and OFF as fast as the refresh rate may allow. The photocurrent may be sampled several times during the ON state, and these readings can be averaged to obtain an ON data point, i_{ON1}. During the subsequent OFF state, the photocurrent may be sampled again several times and averaged down to obtain a single OFF data point i_{OFF1}. In order to remove common mode error, the photocurrent i_{OFF1} may be subtracted from the photocurrent i_{ON1}. This process may be repeated many times resulting in a digital representation of the PPG signal which BP=a+b*PTT may be free of common mode error, such as electronic noise, ambient light induced signal, etc.

For HR measurements, the frequency of the pulsing image may be rather lenient and could be met with even the slowest refreshing displays. For the PTT measurement, however, the requirements may be more stringent. Typical pulse transit time for when a PPG signal is picked off of a user's fingertip is approximately 200 ms. In order to achieve 5% accuracy in the measurement of this delay time, 10 ms of absolute accuracy may be required. In other words, the peak of the PPG signal must be determined with 10 ms of accuracy. In order to achieve this, the photocurrent may be sampled at least three times during the 10 ms, which equates to a 300 Hz sampling rate, meaning a 300 Hz sampling rate for the display. Thus, even if a typical display refreshes relatively fast with a 120 Hz frequency, the accuracy of the PTT measurement may only be 10-15%, resulting in a BP measurement having an error of at least 10-15%.

Ordinary, this error in the BP measurement may be too high to consider the measurement accurate or useful. However, the estimated 10-15% error may be obtained for one ON state and on ONE off state only. The pair of states may last only 66 ms in the case of a 30 Hz refresh rate display. If the BP output data rate is expected to be once per every 15 seconds, the measurement can be repeated approximately 220 times. By averaging down, the random error can be reduced by sqrt(220)=15 times.

The estimates provided above show that the touchscreen display 250 can be run in pulse mode and used as a light source for obtaining a PPG measurement. Error in the BP determination may be dominated by calibration, rather than by errors in PTT measurements (see Eq. 1 below). In some implementations the backlight of the touchscreen display 250 may be augmented by adding infrared (IR) light sources and the PPG measurements may be performed using the IR light.

As described herein, the smartphone device 210 can obtain both PPG and ECG measurements for the user in order to determine the user's BP using PTT techniques. The measurement of the two signals (e.g., PPG and ECG) can be performed simultaneously. Each signal may be time-stamped using the same clock on the smartphone device 210. Alternatively, the time stamp for each signal may be obtained from two difference clocks. For example, from a ECG clock and from a PPG clock, wherein both clocks are synchronized with a system clock. Thus, accurate measurement of the PTT can be obtained. From the PTT data, the BP of the individual can be computed using the following formula and techniques known in the prior art:

BP=a+b*PTT

where a is an intercept and b is a slope of the calibration function.

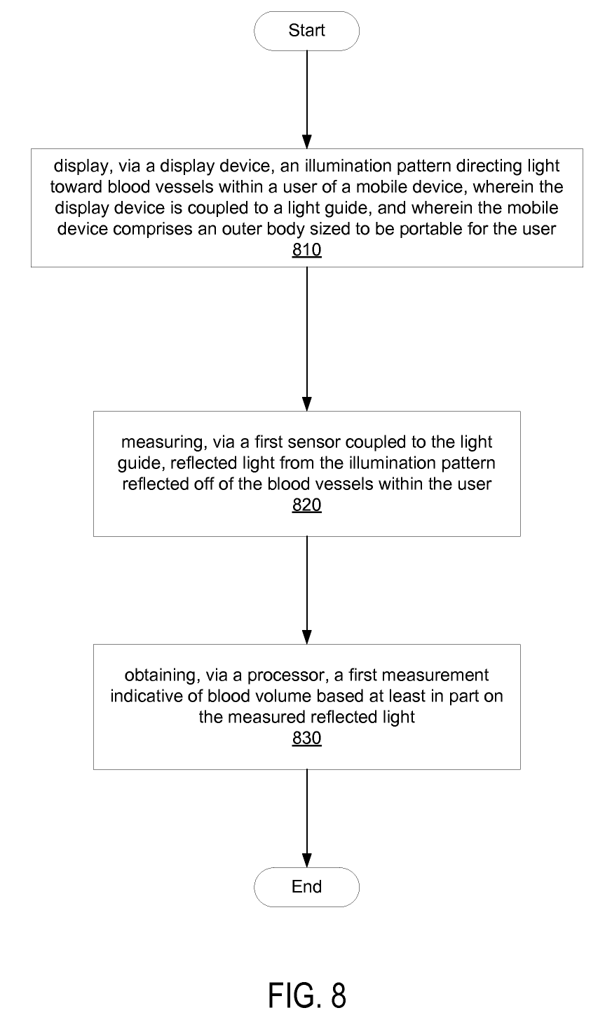

FIG. 8 is a flowchart of a method for obtaining vital measurements, according to some implementations. In block 810, an illumination pattern directing light toward blood vessels within a user of a mobile device may be displayed. The display device may be coupled to a light guide, and the mobile device may include an outer body sized to be portable for the user. The illuminating pattern may be a red or green colored image. The light guide may be a cover glass positioned above the display device having light guiding or light turning features. For example, in FIG. 4, the cover glass functions as a light guide to guide the reflected light reflected off the user's finger toward the light sensor. The light is displayed on the display in the form of an illumination pattern.

In block 820, reflected light from the illumination pattern reflected off of the blood vessels within the user may be measured via a first sensor coupled to the light guide. For example, in FIG. 4, once the reflected light is “guided” toward the light sensor, the light sensor measured the amount of detected light. The light sensor may be a photodiode.

Additionally, a second measurement indicative of heart electrical activity may be obtained via a second sensor coupled to the outer body. The second measurement may of heart electrical activity may be an ECG measurement. For example, in FIG. 3, the two contacts on the side of the phone are used to complete a circuit through the user's body and obtain an ECG measurement. The contacts may be electrodes.

In block 830, a first measurement indicative of changes in blood volume based at least in part on the measured reflected light is obtained. The measurement may be obtained via a processor of a mobile device. For example, in FIG. 3, after the user grabs the device in the appropriate manner, the device may obtain both PPG and ECG measurements for the user. Both the PPG and ECG measurements may be used to determine a PTT, which in turn is used to determine the BP for the user.

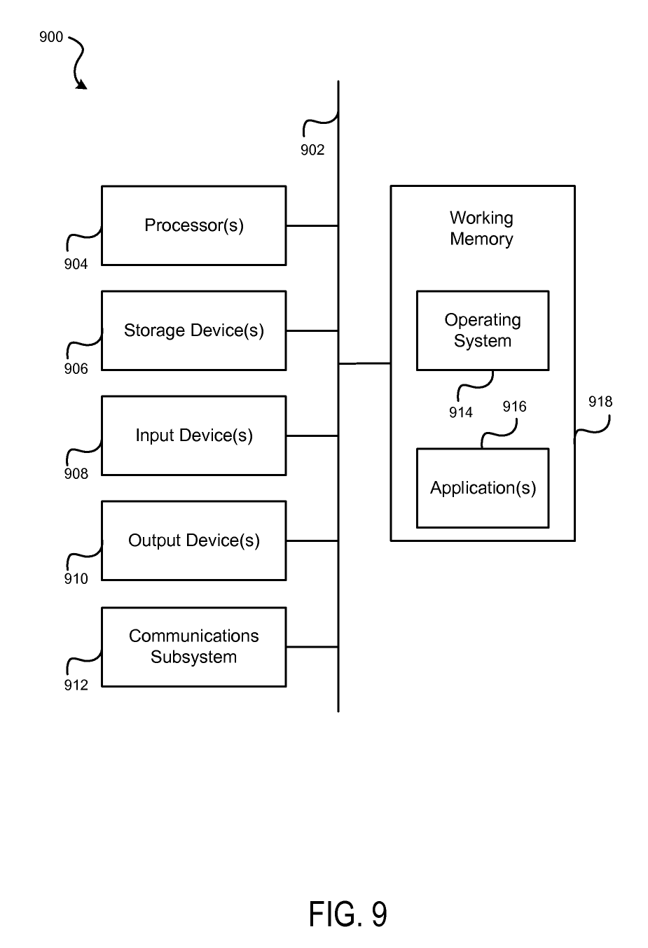

FIG. 9 illustrates an example of a computing system in which one or more embodiments may be implemented. A computer system as illustrated in FIG. 9 may be incorporated as part of the above described computerized device. For example, computer system 900 can represent some of the components of a television, a computing device, a server, a desktop, a workstation, a control or interaction system in an automobile, a tablet, a netbook or any other suitable computing system. A computing device may be any computing device with an image capture device or input sensory unit and a user output device. An image capture device or input sensory unit may be a camera device. A user output device may be a display unit. Examples of a computing device include but are not limited to video game consoles, tablets, smart phones and any other hand-held devices. FIG. 9 provides a schematic illustration of one embodiment of a computer system 900 that can perform the methods provided by various other embodiments, as described herein, and/or can function as the host computer system, a remote kiosk/terminal, a point-of-sale device, a telephonic or navigation or multimedia interface in an automobile, a computing device, a set-top box, a table computer and/or a computer system. FIG. 9 is meant only to provide a generalized illustration of various components, any or all of which may be utilized as appropriate. FIG. 9, therefore, broadly illustrates how individual system elements may be implemented in a relatively separated or relatively more integrated manner. In some embodiments, elements of computer system 900 may be used to implement functionality of the mobile device 100 in FIG. 1.

The computer system 900 is shown comprising hardware elements that can be electrically coupled via a bus 902 (or may otherwise be in communication, as appropriate). The hardware elements may include one or more processors 904, including without limitation one or more general-purpose processors and/or one or more special-purpose processors (such as digital signal processing chips, graphics acceleration processors, and/or the like); one or more input devices 908, which can include without limitation one or more cameras, sensors, a mouse, a keyboard, a microphone configured to detect ultrasound or other sounds, and/or the like; and one or more output devices 910, which can include without limitation a display unit such as the device used in embodiments described herein, a printer and/or the like.

In some implementations of the embodiments described herein, various input devices 908 and output devices 910 may be embedded into interfaces such as display devices, tables, floors, walls, and window screens. Furthermore, input devices 908 and output devices 910 coupled to the processors may form multi-dimensional tracking systems.

The computer system 900 may further include (and/or be in communication with) one or more non-transitory storage devices 906, which can comprise, without limitation, local and/or network accessible storage, and/or can include, without limitation, a disk drive, a drive array, an optical storage device, a solid-state storage device such as a random access memory (“RAM”) and/or a read-only memory (“ROM”), which can be programmable, flash-updateable and/or the like. Such storage devices may be configured to implement any appropriate data storage, including without limitation, various file systems, database structures, and/or the like.

The computer system 900 might also include a communications subsystem 912, which can include without limitation a modem, a network card (wireless or wired), an infrared communication device, a wireless communication device and/or chipset (such as a Bluetooth™ device, an 802.11 device, a Wi-Fi device, a WiMax device, cellular communication facilities, etc.), and/or the like. The communications subsystem 912 may permit data to be exchanged with a network, other computer systems, and/or any other devices described herein. In many embodiments, the computer system 900 will further comprise a non-transitory working memory 918, which can include a RAM or ROM device, as described above.

The computer system 900 also can comprise software elements, shown as being currently located within the working memory 918, including an operating system 914, device drivers, executable libraries, and/or other code, such as one or more application programs 916, which may comprise computer programs provided by various embodiments, and/or may be designed to implement methods, and/or configure systems, provided by other embodiments, as described herein. Merely by way of example, one or more procedures described with respect to the method(s) discussed above might be implemented as code and/or instructions executable by a computer (and/or a processor within a computer); in an aspect, then, such code and/or instructions can be used to configure and/or adapt a general purpose computer (or other device) to perform one or more operations in accordance with the described methods.

A set of these instructions and/or code might be stored on a computer-readable storage medium, such as the storage device(s) 906 described above. In some cases, the storage medium might be incorporated within a computer system, such as computer system 900. In other embodiments, the storage medium might be separate from a computer system (e.g., a removable medium, such as a compact disc), and/or provided in an installation package, such that the storage medium can be used to program, configure and/or adapt a general purpose computer with the instructions/code stored thereon. These instructions might take the form of executable code, which is executable by the computer system 900 and/or might take the form of source and/or installable code, which, upon compilation and/or installation on the computer system 900 (e.g., using any of a variety of generally available compilers, installation programs, compression/decompression utilities, etc.) then takes the form of executable code.

Substantial variations may be made in accordance with specific requirements. For example, customized hardware might also be used, and/or particular elements might be implemented in hardware, software (including portable software, such as applets, etc.), or both. Further, connection to other computing devices such as network input/output devices may be employed. In some embodiments, one or more elements of the computer system 900 may be omitted or may be implemented separate from the illustrated system. For example, the processor 904 and/or other elements may be implemented separate from the input device 908. In one embodiment, the processor is configured to receive images from one or more cameras that are separately implemented. In some embodiments, elements in addition to those illustrated in FIG. 9 may be included in the computer system 900.

Some embodiments may employ a computer system (such as the computer system 900) to perform methods in accordance with the disclosure. For example, some or all of the procedures of the described methods may be performed by the computer system 900 in response to processor 904 executing one or more sequences of one or more instructions (which might be incorporated into the operating system 914 and/or other code, such as an application program 916) contained in the working memory 918. Such instructions may be read into the working memory 918 from another computer-readable medium, such as one or more of the storage device(s) 906. Merely by way of example, execution of the sequences of instructions contained in the working memory 918 might cause the processor(s) 904 to perform one or more procedures of the methods described herein.

The terms “machine-readable medium” and “computer-readable medium,” as used herein, refer to any medium that participates in providing data that causes a machine to operate in a specific fashion. In some embodiments implemented using the computer system 900, various computer-readable media might be involved in providing instructions/code to processor(s) 904 for execution and/or might be used to store and/or carry such instructions/code (e.g., as signals). In many implementations, a computer-readable medium is a physical and/or tangible storage medium. Such a medium may take many forms, including but not limited to, non-volatile media, volatile media, and transmission media. Non-volatile media include, for example, optical and/or magnetic disks, such as the storage device(s) 906. Volatile media include, without limitation, dynamic memory, such as the working memory 918. Transmission media include, without limitation, coaxial cables, copper wire and fiber optics, including the wires that comprise the bus 902, as well as the various components of the communications subsystem 912 (and/or the media by which the communications subsystem 912 provides communication with other devices). Hence, transmission media can also take the form of waves (including without limitation radio, acoustic and/or light waves, such as those generated during radio-wave and infrared data communications).

Common forms of physical and/or tangible computer-readable media include, for example, a floppy disk, a flexible disk, hard disk, magnetic tape, or any other magnetic medium, a CD-ROM, any other optical medium, punchcards, papertape, any other physical medium with patterns of holes, a RAM, a PROM, EPROM, a FLASH-EPROM, any other memory chip or cartridge, a carrier wave as described hereinafter, or any other medium from which a computer can read instructions and/or code.

Various forms of computer-readable media may be involved in carrying one or more sequences of one or more instructions to the processor(s) 1004 for execution. Merely by way of example, the instructions may initially be carried on a magnetic disk and/or optical disc of a remote computer. A remote computer might load the instructions into its dynamic memory and send the instructions as signals over a transmission medium to be received and/or executed by the computer system 900. These signals, which might be in the form of electromagnetic signals, acoustic signals, optical signals and/or the like, are all examples of carrier waves on which instructions can be encoded, in accordance with various embodiments described herein.

The communications subsystem 912 (and/or components thereof) generally will receive the signals, and the bus 902 then might carry the signals (and/or the data, instructions, etc. carried by the signals) to the working memory 918, from which the processor(s) 904 retrieves and executes the instructions. The instructions received by the working memory 918 may optionally be stored on a non-transitory storage device 906 either before or after execution by the processor(s) 904.

The methods, systems, and devices discussed above are examples. Various configurations may omit, substitute, or add various procedures or components as appropriate. For instance, in alternative configurations, the methods may be performed in an order different from that described, and/or various stages may be added, omitted, and/or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

Also, configurations may be described as a process which is depicted as a flow diagram or block diagram. Although each may describe the operations as a sequential process, many of the operations can be performed in parallel or concurrently. In addition, the order of the operations may be rearranged. A process may have additional steps not included in the figure. Furthermore, examples of the methods may be implemented by hardware, software, firmware, middleware, microcode, hardware description languages, or any combination thereof. When implemented in software, firmware, middleware, or microcode, the program code or code segments to perform the necessary tasks may be stored in a non-transitory computer-readable medium such as a storage medium. Processors may perform the described tasks.

Having described several example configurations, various modifications, alternative constructions, and equivalents may be used without departing from the spirit of the disclosure. For example, the above elements may be components of a larger system, wherein other rules may take precedence over or otherwise modify the application of the embodiments. Also, a number of steps may be undertaken before, during, or after the above elements are considered.

What is claimed is:

1. A mobile device for obtaining vital measurements, comprising:
an outer body sized to be portable for a user of the mobile device;
a processor contained within the outer body;
a display coupled to a light guide, the display configured to display an illumination pattern directing light toward blood vessels within the user; and
at least one first sensor coupled to the light guide, the at least one first sensor configured to measure reflected light from the illumination pattern reflected off of the blood vessels within the user, wherein the processor is configured to obtain a first measurement indicative of changes in blood volume based at least in part on the measured reflected light.

2. The mobile device of claim 1, further comprising at least one second sensor coupled to the outer body, the at least one second sensor configured to obtain a second measurement indicative of heart electrical activity.

3. The mobile device of claim 2, wherein the second measurement indicative of heart electrical activity comprises an electrocardiography (ECG) measurement.

4. The mobile device of claim 2, wherein the at least one second sensor comprises at least a first electrode and a second electrode, and wherein a portion of the user's body completes a circuit between the first electrode and the second electrode.

5. The mobile device of claim 2, wherein the processor is further configured to facilitate generation of a blood pressure value based on the first measurement and the second measurement.

6. The mobile device of claim 1, wherein the at least one first sensor comprises a photodiode.

7. The mobile device of claim 1, wherein the at least one first sensor is positioned at an edge of the display.

8. The mobile device of claim 1, wherein the first measurement indicative of changes in blood volume comprises a photoplethysmography (PPG) measurement.

9. The mobile device of claim 1, wherein the mobile device is at least one of a smartphone device or a watch.

10. A method for obtaining vital measurements, comprising:
displaying, via a display device, an illumination pattern directing light toward blood vessels within a user of a mobile device, wherein the display device is coupled to a light guide, and wherein the mobile device comprises an outer body sized to be portable for the user;
measuring, via a first sensor coupled to the light guide, reflected light from the illumination pattern reflected off of the blood vessels within the user; and
obtaining, via a processor, a first measurement indicative of changes in blood volume based at least in part on the measured reflected light.

11. The method of claim 10, further comprising obtaining, via a second sensor coupled to the outer body, a second measurement indicative of heart electrical activity.

12. The method of claim 11, wherein the second sensor comprises at least a first electrode and a second electrode, and wherein a portion of the user's body completes a circuit between the first electrode and the second electrode.

13. The method of claim 11, wherein the second measurement indicative of heart electrical activity comprises an electrocardiography (ECG) measurement.

14. The method of claim 11, further comprising facilitating, via the processor, generation of a blood pressure value based on the first measurement and the second measurement.

15. The method of claim 10, wherein the first sensor comprises a photodiode.

16. The method of claim 10, wherein the first sensor is positioned at an edge of the display.

17. The method of claim 10, wherein the first measurement indicative of changes in blood volume comprises a photoplethysmography (PPG) measurement.

18. The method of claim 10, wherein the mobile device is at least one of a smartphone device or a watch.

19. An apparatus for obtaining vital measurements, comprising:
means for displaying, via a display device, an illumination pattern directing light toward blood vessels within a user of a mobile device, wherein the display device is coupled to a light guide, and wherein the mobile device comprises an outer body sized to be portable for the user;
means for measuring, via a first sensor coupled to the light guide, reflected light from the illumination pattern reflected off of the blood vessels within the user; and
means for obtaining, via a processor, a first measurement indicative of changes in blood volume based at least in part on the measured reflected light.

20. The apparatus of claim 19, further comprising means for obtaining, via a second sensor coupled to the outer body, a second measurement indicative of heart electrical activity.

21. The apparatus of claim 20, wherein the second sensor comprises at least a first electrode and a second electrode, and wherein a portion of the user's body completes a circuit between the first electrode and the second electrode.

22. The apparatus of claim 20, wherein the second measurement indicative of heart electrical activity comprises an electrocardiography (ECG) measurement.

23. The apparatus of claim 20, further comprising means for facilitating, via the processor, generation of a blood pressure value based on the first measurement and the second measurement.

24. The apparatus of claim 19, wherein the first sensor comprises a photodiode.

25. The apparatus of claim 19, wherein the first sensor is positioned at an edge of the display.

26. The apparatus of claim 19, wherein the first measurement indicative of changes in blood volume comprises a photoplethysmography (PPG) measurement.

27. The apparatus of claim 19, wherein the mobile device is at least one of a smartphone device or a watch.

28. One or more non-transitory computer-readable media storing computer-executable instructions for obtaining vital measurements that, when executed, cause one or more computing devices included in a mobile device to:
display, via a display device, an illumination pattern directing light toward blood vessels within a user of a mobile device, wherein the display device is coupled to a light guide, and wherein the mobile device comprises an outer body sized to be portable for the user;
measure, via a first sensor coupled to the light guide, reflected light from the illumination pattern reflected off of the blood vessels within the user; and
obtain, via a processor, a first measurement indicative of changes in blood volume based at least in part on the measured reflected light.

29. The non-transitory computer-readable media of claim 28, further comprising obtaining, via a second sensor coupled to the outer body, a second measurement indicative of heart electrical activity.

30. The non-transitory computer-readable media of claim 29, wherein the second sensor comprises at least a first electrode and a second electrode, and wherein a portion of the user's body completes a circuit between the first electrode and the second electrode.

31. The non-transitory computer-readable media of claim 29, wherein the second measurement indicative of heart electrical activity comprises an electrocardiography (ECG) measurement.

32. The non-transitory computer-readable media of claim 29, wherein the instructions, when executed, further cause the one or more computing devices to facilitate, via the processor, generation of a blood pressure value based on the first measurement and the second measurement.

33. The non-transitory computer-readable media of claim 28, wherein the first sensor comprises a photodiode.

34. The non-transitory computer-readable media of claim 28, wherein the first sensor is positioned at an edge of the display.

35. The non-transitory computer-readable media of claim 28, wherein the first measurement indicative of changes in blood volume comprises a photoplethysmography (PPG) measurement.

36. The non-transitory computer-readable media of claim 28, wherein the mobile device is at least one of a smartphone device or a watch.
